(19) United States
(12) Patent Application Publication
(10) Pub. No.: US 2012/0309947 A1
(43) Pub. Date: Dec. 6, 2012

(54) METHOD AND SYSTEM FOR CONTINUOUS SEPARATION AND PURIFICATION OF GINSENOSIDES

(76) Inventors: Zer-Ran YU, Chiayi City (TW); Be-Jen Wang, Chiayi City (TW)

(21) Appl. No.: 13/151,895

(22) Filed: Jun. 2, 2011

(51) Int. Cl. C07J 75/00; B01D 11/04

(52) U.S. Cl. 536/6; 422/260

(57) ABSTRACT

The present invention provides a method and system for continuous separation and purification of ginsenosides, whereby supercritical fluid technology is used to feed Ginseng extract liquor and supercritical solvent continuously into a separation tank at 20-30 Mpa and 40-60° C., such that ginsenosides can be separated from Ginseng extract liquor; then, ginsenosides are continuously fed to a purifying tank for obtaining highly pure ginsenosides; this system comprising: a holding tank, used to accommodate Ginseng extract liquor; a separation tank, connected with the holding tank, fitted with an electric heater, and used to separate ginsenosides from Ginseng extract liquor; a supercritical fluid vessel, connected with the separation tank to provide supercritical fluid; a high-pressure metering pump, connected between the supercritical fluid vessel and separation tank; a reactant metering pump, connected between the holding tank and separation tank; a precooler, connected between the supercritical fluid vessel and high-pressure metering pump; two preheaters, connected separately between the high-pressure metering pump, reactant metering pump and separation tank; a purifying tank, connected with the separation tank, fitted with an electric heater, and used to purify ginsenosides; two temperature controllers, connected separately with the electric heater in the separation tank and purifying tank.

### BACKGROUND OF INVENTION

1. Field of the Invention

The present invention relates generally to a ginsenosides extraction technology, and more particularly to a method and system for continuous separation and purification of ginsenosides.

2. Description of Related Art

Ginseng refers to the root of Araliaceae perennial herb: Panax ginseng C. A. Meyer, which was recorded in Shennong's herbal classic as a top-grade drug. Modern pharmacological studies prove that, Ginseng can regulate the central nervous system and immune function, anti-oxidation and anti-inflammation, reduce blood sugar and improve digestion and metabolic functions, etc. Among various effective constituents of Ginseng, ginsenosides including Rb1, Rb2, Rc, Rd, Re, Rg1 and Rh2 are response for its physiological activities, of which Rb1 can lower the blood lipid, anti-oxidation, anti-inflammation and adjust the immune functions, whilst Rg1 is believed to be capable of stimulating central nervous system, resisting fatigue, improving memory and learning functions as well as promoting angiogenesis; and Rh2 is capable of restraining proliferation of cancer cells and tumor.

With regard to common ginsenosides extraction technologies, as shown in U.S. Pat. No. 7,759,101, ginsenosides glycosidase could be used to increase the content of ginsenosides, or in U.S. Pat. No. 7,371,416, a 2-h reaction is made at working pressure of 1.4-1.5 kg/cm², and temperature of 126-130° C.; however, most of impurities in Ginseng extracts could not be removed to improve its purity. In U.S. Pat. No. 7,884,195, column chromatography is employed to increase the content of ginsenosides, but safety considerations are required due to bulk use of organic solvents.

### CONTENT OF THE INVENTION

The main purpose of the present invention is to provide a method and system for continuous separation and purification of ginsenosides, whereby supercritical fluid technology and continuous separation & purification system are used to separate and purify ginsenosides from Ginseng extract liquor by controlling the operating temperature and pressure, without the problems of solvent residues and toxicity.

To this end, the present invention provides a method for continuous separation and purification of ginsenosides, which allows to feed Ginseng extract liquor and supercritical solvent continuously into a separation tank at a preset flow rate, wherein ginseng extract liquor is separated to obtain ginsenosides; then, ginsenosides is fed to a purifying tank for purification; the operating conditions of the separation tank and purifying tank are: pressure of 20-30 MPa and temperature of 40-60° C.

The present invention provides a system for continuous separation and purification of ginsenosides, which comprises a holding tank to accommodate Ginseng extract liquor; a separation tank, connected with the holding tank, equipped with an electric heater and used to separate ginsenosides from Ginseng extract liquor; a supercritical fluid vessel, connected with the separation tank to provide supercritical fluid; a high-pressure metering pump, connected between the supercritical fluid vessel and separation tank; a reactant metering pump, connected between the holding tank and separation tank; a precooler, connected between the supercritical fluid vessel and high-pressure metering pump; two preheaters, connected separately between the high-pressure metering pump, reactant metering pump and separation tank; a purifying tank, connected with the separation tank, equipped with an electric heater and used to purify the ginsenosides; two temperature controllers, connected separately with the electric heater in the separation tank and purifying tank.

### BRIEF DESCRIPTION OF THE DRAWINGS

FIG. 1 is a sketch diagram of the continuous separation system of the preferred embodiment of the present invention; and

FIG. 2 is a diagram, showing the relationship of pressure, temperature and selectivity.

### DETAILED DESCRIPTION OF THE INVENTION

The following is a typical preferred embodiment of the present invention, which is described below in conjunction with the accompanying drawings:

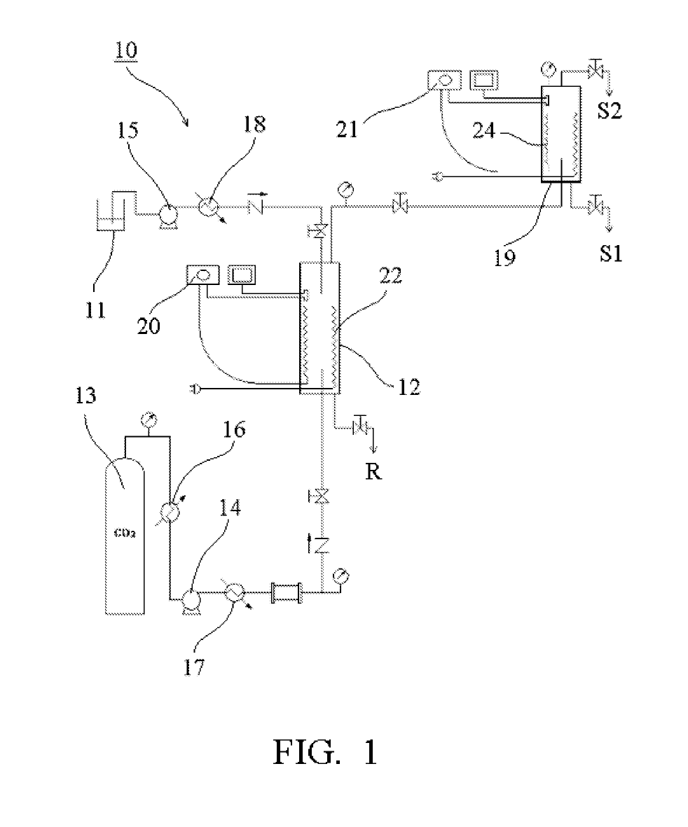

FIG. 1 depicts a preferred embodiment of a method of the present invention for continuous separation and purification of ginsenosides, wherein a system 10 is used to prepare continuously ginsenosides with supercritical fluid technology; said system 10 comprises: a holding tank 11, a separation tank 12, a supercritical fluid vessel 13, a high-pressure metering pump 14, a reactant metering pump 15, a precooler 16, two preheaters 17, 18, a purifying tank 19 and a temperature controller 20, 21 as well as valves. The holding tank 11 is used to accommodate Ginseng extract liquor; the separation tank 12 connected with the holding tank 11 is composed of stainless steel tank body and monomer body, wherein an electric heater 22 is placed; the supercritical fluid vessel 13, referred to as a CO₂ steel cylinder, is connected with the separation tank 12 to provide CO₂ supercritical fluid; the high-pressure metering pump 14 is connected between the supercritical fluid vessel 13 and separation tank 12; the reactant metering pump 15 is connected between the holding tank 11 and separation tank 12; the precooler 16 is connected between the supercritical fluid vessel 13 and high-pressure metering pump 14; two preheaters 17, 18 are connected separately between the high-pressure metering pump 14, reactant metering pump 15 and separation tank 12; the purifying tank 19 connected with the separation tank 12 consists of a tank body and silicon monomer (Si-60 monomer) set within the tank body, wherein an electric heater 24 is placed; two temperature controllers 20, 21 are connected separately with the electric heaters 22, 24 within the separation tank 12 and purifying tank 19.

According to the method of the present invention for continuous separation and purification of ginsenosides, ginseng extract liquor and supercritical solvent are fed at a preset flow rate into the separation tank 12, so as to separate Ginseng extract liquor in the separation tank 12; then ginsenosides are taken out and fed to the purifying tank 19 for purification. In detail, the supercritical fluid vessel 13 is firstly opened, then high-pressure metering pump 14 and valves are used to control the pressure of the separation tank 12, and the temperature controller 20 is used to control the temperature of the separation tank 12, with the operating conditions: 40-60° C., 20-30 MPa, flow rate of supercritical fluid 1 L/hr, flow rate of ginseng extract liquor 0.3-0.5 L/hr, and concentration of 1-5% ginseng extract liquor; then the reactant metering pump 15 is started to add ginseng extract liquor into the separation tank 12 in a controlled way; so ginseng extract liquor can be separated in the separation tank 12 to form ginsenosides and other constituents (e.g.: polysaccharides). Ginsenosides is separated at top of separation tank 12.

After ginseng extract liquor is separated in the separation tank 12, then ginsenosides is fed to the purifying tank 19 for purification; the samples of purified ginsenosides are collected at top (S2) of the purifying tank 19, and flushed reversely 5 minutes with water to separate highly pure ginsenosides samples and collect them at bottom of the purifying tank (S1).

As listed in Table 1, 9 groups of examples are designed experimentally based on orthogonal array of four factors: separation temperature (40, 50, 60° C.), pressure (20, 25, 30 MPa), Ginseng feed concentration (1.0, 2.5, 5.0%) and Ginseng feed rate (0.3, 0.4, 0.5 L/hr).

TABLE 1

| | | | Ginseng feed | Feed |
|---|---|---|---|---|
| | Temperature | Pressure | concentration | rate |
| Examples | (° C.) | (MPa) | (%) | (L/hr) |
| 1 | 50 | 30 | 1.0 | 0.3 |
| 2 | 60 | 20 | 1.0 | 0.4 |
| 3 | 40 | 30 | 2.5 | 0.4 |
| 4 | 60 | 25 | 2.5 | 0.3 |
| 5 | 50 | 20 | 2.5 | 0.5 |
| 6 | 60 | 30 | 5.0 | 0.5 |
| 7 | 40 | 20 | 5.0 | 0.3 |
| 8 | 40 | 25 | 1.0 | 0.5 |
| 9 | 50 | 25 | 5.0 | 0.4 |

In detail, ginsenosides samples R at bottom of the separation tank 12 and ginsenosides samples S2 at top of the purifying tank 19 are collected and analyzed to determine total ginsenosides, and calculate the separation efficiency (Ks=total ginsenosides at top of the purifying tank÷total ginsenosides at bottom of the separation tank); the separated value is subject to variable analysis by linear regression of SPSS statistical software. The statistical analysis result indicates that: the concentration of Ginseng extract liquor (1.0, 2.5, 5.0%) and Ginseng feed rate (0.3, 0.4, 0.5 L/hr) do not affect the separation efficiency of ginsenosides (p>0.05), but the operating pressure and temperature will affect the separation efficiency of ginsenosides (p<0.05).

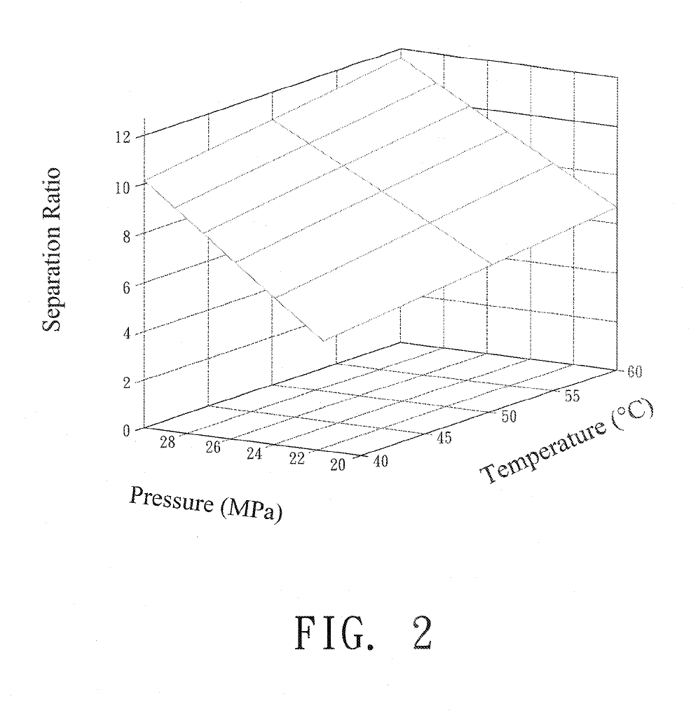

In addition, the data were analyzed using Response Surface Methodology of SPSS statistical software. The statistical results indicate that, as shown in FIG. 2, when the pressure is higher (i.e. up to 30 MPa) and temperature is at 55-60° C., the separation efficiency reaches 11-12. For instance, if Ginseng solution containing approx. 5.0% ginsenosides (sample at position F in FIG. 1) is continuously separated and purified in the separation tank 12 and purifying tank 19 under aforementioned operating conditions, the analytical results indicate that: Ginseng solution containing approx. 50-60% ginsenosides could be purified at top of the purifying tank 19 (sample at position S2 shown in FIG. 1), then flushed reversely with water at top of the purifying tank to separate samples containing approx. 30-35% ginsenosides, and collect them at bottom of the purifying tank 19 (S1).

According to the method and system of the present invention for continuous separation and purification of ginsenosides, ginseng extract liquor can be separated and purified into ginsenosides by using supercritical fluid technology and continuous separation & purification system; as compared with conventional extraction method, the present invention enables separation and purification of ginsenosides by only controlling the operating temperature and pressure, without the problems of solvent residue and toxicity; moreover, ginsenosides can be easily separated from CO₂ solvents while the entire system is operated continuously.

What is claimed is:

1. A method for continuous separation and purification of ginsenosides, whereby supercritical fluid technology is used to feed Ginseng extract liquor and supercritical solvent into a separation tank at a preset flow rate under operating conditions of 20-30 Mpa and 40-60° C., then Ginseng extract liquor can be separated into ginsenosides in the separation tank, and then fed continuously to a purifying tank for obtaining highly pure ginsenosides.

2. The method defined in claim 1, wherein said supercritical solvent is CO₂ fluid in supercritical state.

3. The method defined in claim 1, wherein Ginseng extract liquor of 1-5% is fed into the separation tank at a rate of 0.3-0.5 L/hr, and supercritical solvent fed into the separation tank at a rate of 1 L/hr.

4. The method defined in claim 1, wherein ginsenosides are separated at top of the separation tank.

5. The method defined in claim 1, wherein ginsenosides are flushed reversely with water in the purifying tank.

6. The method defined in claim 5, wherein the samples are flushed reversely 5 minutes with water in the purifying tank to obtain highly pure ginsenosides at bottom of the purifying tank.

7. The method defined in claim 1, wherein the optimum operating conditions of the separation tank include: pressure of 30 Mpa and temperature of 55-60° C.

8. The system defined in claim 1, wherein it comprising:
a holding tank, used to accommodate Ginseng extract liquor;
a separation tank, connected with the holding tank, fitted with an electric heater, and used to separate ginsenosides from Ginseng extract liquor;
a supercritical fluid vessel, connected with the separation tank to provide supercritical fluid; a high-pressure metering pump, connected between the supercritical fluid vessel and separation tank; a reactant metering pump, connected between the holding tank and separation tank;
a precooler, connected between the supercritical fluid vessel and high-pressure metering pump; two preheaters, connected separately between the high-pressure metering pump, reactant metering pump and separation tank;
a purifying tank, connected with the separation tank, fitted with an electric heater, and used to purify ginsenosides; and
two temperature controllers, connected separately with the electric heaters in the separation tank and purifying tank.

9. The system defined in claim 8, wherein said separation tank consists of a tank body and stainless steel monomer body within the tank body; the electric heater is set within the tank body.

10. The system defined in claim 8, wherein said purifying tank consists of a tank body and silicon monomer within the tank body; the electric heater is set within the tank body.

11. The system defined in claim 8, wherein said supercritical fluid vessel is used to accommodate is CO₂ fluid in supercritical state.
